(19) United States
(12) Patent Application Publication
(10) Pub. No.: US 2025/0098939 A1
(43) Pub. Date: Mar. 27, 2025

(54) Wireless Imaging System

(71) Applicant: Lazurite Holdings LLC, Cleveland Heights, OH (US)

(72) Inventors: Laimis Kazimieras BELZINSKAS, Kirtland, OH (US); Eugene MALINSKIY, Mayfield Village, OH (US); Ilya MALINSKIY, Cleveland Heights, OH (US); Howard FEIN, Cleveland Heights, OH (US); Stephen LATHAM, Sun Prairie, WI (US); Craig CONNER, Madison, WI (US)

(21) Appl. No.: 18/832,406

(22) PCT Filed: Jan. 23, 2023
(86) PCT No.: PCT/US23/61080

§ 371 Date: Jul. 23, 2024

Related U.S. Application Data
(60) Provisional application No. 63/302,506, filed on Jan. 24, 2022.

(51) Int. Cl. A61B 1/00; A61B 1/06

(52) U.S. Cl. CPC A61B 1/00016; A61B 1/00052; A61B 1/00114; A61B 1/0653; A61B 1/0676

(57) ABSTRACT

A wireless imaging device including a head unit configured to couple to a scope. The head unit having a housing with an internal cavity configured to receive a battery. The wireless imaging device includes a flexible cable having a proximal end and distal end, the flexible cable coupled to the head unit at the proximal end. The wireless imaging device further includes a light engine having a first end and a second end opposite the first end. The light engine is coupled to the distal end of the flexible cable at the first end and configured to be removably coupled to a scope at the second end such that when the light engine is coupled to the scope, the light engine and the scope are freely rotatable relative to the head unit.

### CROSS-REFERENCE TO RELATED APPLICATIONS

This application claims the benefit of U.S. Provisional Patent Application No. 63/302,506 filed Jan. 24, 2022, entitled “Wireless Imaging System”, which is incorporated by reference herein in its entirety.

### TECHNICAL FIELD

The present disclosure generally relates to a wireless imaging system, and more particularly, a wireless medical imaging system such as arthroscopic and endoscopic devices.

### SUMMARY

One embodiment of the present disclosure provides a wireless imaging device having a head unit configured to couple to a scope, the head unit having a housing with an internal cavity configured to receive a battery, a flexible cable having a proximal end and a distal end, the proximal end of the flexible cable being coupled to the head unit, and a light engine having a first end coupled to the distal end of the flexible cable and a second end opposite the first end. The light engine is configured to be removably coupled to a scope at the second end such that when the light engine is coupled to the scope, the light engine and the scope are freely rotatable relative to the head unit.

In some embodiments, the light engine includes a light converter comprising a plurality of phosphor particles volumetrically disposed within a substrate. The light engine may include an optical element and a window such that the light converter is disposed between the optical element and the window of the light engine. In some embodiments, light converter is a disk.

In some embodiments, the head unit includes a first end and a second end opposite the first end, the first end having a planar front face. The planar front face may include a power cable port and a mount, the power cable port being co-planar with the mount and coupled to the flexible cable. The planar front face may be disposed opposite the internal cavity.

In some embodiments, the wireless imaging device further includes a removable battery sized and shaped to be inserted into the internal cavity. The removable battery may include an exposed distal portion that is exposed and flush with the housing when the removable battery is disposed within the internal cavity. The head unit may include a coupling element configured to engage the removable battery when the removable battery is inserted into the internal cavity.

In some embodiments, the head unit includes a mount configured to couple to one or more of C-mount scopes, eye-piece scopes, and direct view scopes.

In some embodiments, the head unit includes a display screen configured to display an image of a target area viewed by the scope.

In some embodiments, the light engine includes a volumetric converter, an optical element, a window, and an opening arranged in series along a central axis. The light engine may include an outer shell, the outer shell configured to maintain the light engine at a temperature under approximately 45° C.

In some embodiments, the wireless imaging device further includes at least one antenna disposed within the head unit, the at least one antenna configured to wirelessly communicate with a remote receiver.

In some embodiments, the light engine includes an illumination source generating a first light having a first wavelength, the first light being converted by a volumetric converter to a second light having a second wavelength different than the first wavelength. In some embodiments, the light engine includes an outer shell and an opening extending at least partially through the outer shell along a central axis.

Another embodiment of the present disclosure provides a wireless imaging device having a head unit configured to couple to a scope, the head unit having a housing with an internal cavity configured to receive a battery, a first end having a planar face including a power cable port and a mount configured to receive a scope, and a second end being opposite the first end and having the internal cavity, a flexible cable having a proximal end and distal end, the proximal end of the flexible cable being coupled to the head unit at the power cable port, and a light engine coupled to the distal end of the flexible cable, the light engine being configured to be removably coupled to the scope such that when the light engine is coupled to the scope, the light engine and the scope are freely rotatable relative to the head unit. The light engine includes a volumetric converter, an optical element, a window, and an opening arranged in series along a central axis. The light engine also includes an illumination source generating a first light having a first wavelength, the first light being converted by the volumetric converter to a second light having a second wavelength different than the first wavelength.

Another embodiment of the present disclosure provides an imaging system having a wireless imaging device having a head unit configured to removable couple to a scope, the head unit including a proximal end and distal end, the proximal end of the head unit including a power cable port coupled to a flexible cable and the distal end of the head unit including an internal cavity configured to receive a battery, the wireless imaging device including a light engine having a first end coupled to the head unit via the flexible cable and a second end opposite the first end configured to couple to the removable scope, wherein the wireless imaging device includes one or more antennas disposed within the head unit, and a receiver disposed remote to the wireless imaging device, the receiver being in wireless communication with the one or more antennas and configured to wirelessly receive imaging data from the one or more antennas.

In some embodiments, the imaging system further includes a display device in communication with the receiver, the display device having a display screen configured to display the imaging data.

### BRIEF DESCRIPTION OF THE DRAWINGS

The foregoing summary, as well as the following detailed description of embodiments of the wireless imaging system, will be better understood when read in conjunction with the appended drawings of exemplary embodiments. It should be understood, however, that the invention is not limited to the precise arrangements and instrumentalities shown.

In the drawings:

FIG. 1 is a left side view of a wireless imaging device coupled to a portion of an endoscope in accordance with an exemplary embodiment of the present disclosure shown with the battery removed;

FIG. 2 is a right side view of the wireless imaging device of FIG. 1 with a battery fully inserted;

FIG. 3 is a front perspective view of the wireless imaging device of FIG. 1 with the endoscope decoupled;

FIG. 4 is a rear view of the wireless imaging device of FIG. 1 with the battery removed;

FIG. 5 is a cross-sectional view of a light engine of the wireless imaging device of FIG. 1;

FIG. 6A is a front perspective view of a battery charging device for the batteries for use with the wireless imaging device of FIG. 1 in accordance with an exemplary embodiment of the present disclosure;

FIG. 6B is a top view of the battery charging device of FIG. 6A with the batteries removed;

FIG. 7A is a front view of a receiver for use with the wireless imaging device of FIG. 1 in accordance with an exemplary embodiment of the present disclosure;

FIG. 7B is a rear view of the receiver of FIG. 7A;

FIG. 8 is a display screen of a display device in communication with the wireless imaging device of FIG. 1 in accordance with an exemplary embodiment of the present disclosure;

FIG. 9A is a display screen of a display device in communication with the wireless imaging device of FIG. 1 in accordance with an exemplary embodiment of the present disclosure;

FIG. 9B is a display screen of a display device in communication with the wireless imaging device of FIG. 1 in accordance with an exemplary embodiment of the present disclosure; and

FIG. 10 is a front perspective view of a mounting bracket used with a receiver in accordance with an exemplary embodiment of the present disclosure.

### DETAILED DESCRIPTION

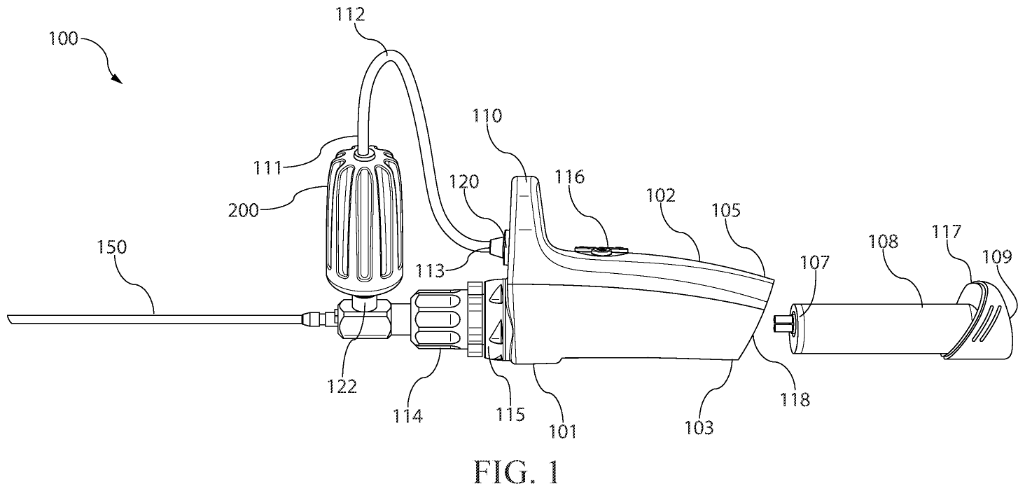

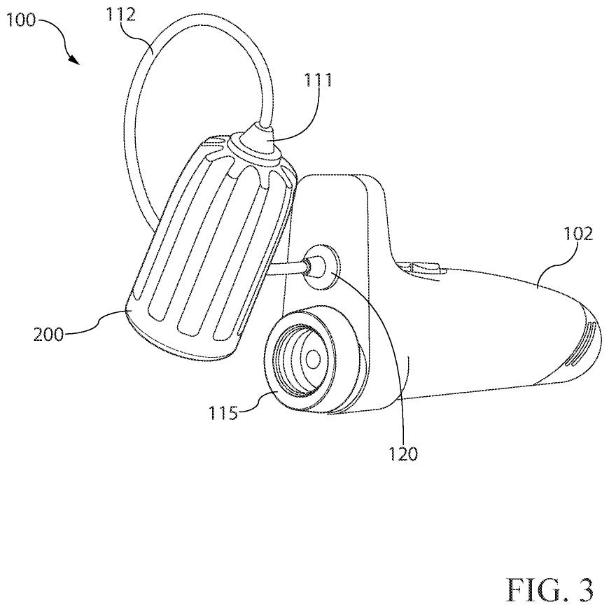

Exemplary embodiments of the present disclosure provide a wireless imaging device. Referring to FIGS. 1-10, there is shown a wireless imaging system. The wireless imaging system may include a wireless imaging device, generally designated 100. The wireless imaging system may further include a receiver (e.g., receiver 400) and a battery charging device (e.g., battery charging device 300) configured to charge a battery (e.g., battery 108). In use, wireless imaging device (“device”) 100 is a wireless device that is configured to provide light to a target area and capture images and/or videos from the target area. Device 100 may be configured for use in different industries and practices such as medicine, dentistry, construction, automotive, aeronautics, or any other type of industry. Device 100 may be wireless such that it allows for easier maneuverability and portability when using device 100. Further, device 100 being wireless may reduce accidents or mistakes due to the elimination of wires that would serve as a trip or fall hazard.

In some embodiments, device 100 includes a scope or optical device (e.g., scope 150) coupled to a head unit (e.g., head unit 102) to aid in visualization. Device 100 may include a medical scope (e.g., endoscope, arthroscope, bronchoscope). Device 100 may be configured to work with most standard medical scopes such that the head unit of device 100 can be coupled to most standard medical scopes without the need for additional adapters or components. Additionally, device 100 may be configured to allow for the use of adapters to assist device 100 in coupling to medical scopes. Device 100 may be configured to be used for endoscopy, arthroscopy, bronchoscopy, gastroscopy, colonoscopy, cystoscopy, or any other type of procedure that requires imaging. However, device 100 may include a head unit configured to couple to non-medical scopes such as borescopes, fiberscopes, videoscopes, or any other type of scope.

In some embodiments, device 100 is a wireless medical imaging device. For example, device 100 may be a wireless medical imaging device that allows a user to diagnose and treat a patient by viewing a target area within a patient. Device 100 may allow for minimally invasive procedures to be provided more easily due to device 100 being wireless. For example, device 100 may be wireless such that no cords are extending from device 100 to a tower or other device. This results in an operating room (OR) having more usable space as less space is taken up by towers and cables, or it may result in a more efficient or smaller room layout. Device 100 being wireless may allow for easier maneuverability of device 100 during use resulting in faster and better treatment of patients. Further, device 100 being wireless allows for faster set-up time, use time, and break-down time for surgical procedures. Device 100 being wireless also may decrease the number of individuals required to perform the procedures or assist with performance of the procedures. For example, device 100 being wireless may reduce the number of nurses or technicians needed to perform the procedure. Additionally, reprocessing, cleaning, and sterilization time may also be reduced due to the lack of wires and the smaller footprint of device 100 compared to conventional imaging systems. The improvements herein may save considerable time and expenses for the user of device 100 (e.g., medical provider or hospital).

In some embodiments, the portability and wireless nature of device 100 allows for device 100 to be used anywhere without the need for a medical setting. Since device 100 can be used anywhere and may not require a medical setting, a patient may receive medical care without having to be moved to another location. In some embodiments, device 100 being wireless allows it to be used at many different locations such as in/out-patient surgical centers, physician offices, nursing homes/long term care facilities, and mobile clinics and surgical centers that can go to a patient directly. For example, a bed-bound patient at a long-term facility who is suffering from an injury could be treated at that facility and rapidly be provided rehabilitation services instead of spending additional time at a hospital operating room or having to suffer from difficult transportation. Further, device 100 may allow for the creation of mobile diagnostic and surgical suites that could provide minimally invasive surgery next to the homes of their patients thereby decreasing the pre- and post-surgical time, and quickly returning the patient to a comfortable environment. Additionally, due to the compact size and easy portability of device 100, device 100 may allow for the use of advanced imaging and surgical procedures in austere environments that do not have access to modern hospitals such as, for example, developing countries, military operations, or natural disaster areas.

Referring to FIGS. 1-4, device 100 may include head unit 102 and light source or light engine 200. Head unit 102 of device 100 may be configured to couple to scope 150. In some embodiments, scope 150 is coupled to head unit 102 and light engine 200. In some embodiments, scope 150 is a laparoscope, ENT scope, an arthroscope, or any other type of medical imaging scope. Head unit 102 may be coupled to light engine 200 via power cable (“cable”) 112. In some embodiments, head unit 102 is configured to receive an image from scope 150 that is illuminated via light from light engine 200. For example, light engine 200 may be configured to output light that travels through scope 150 to illuminate a target area. Light from the target area may reflect back through scope 150 to head unit 102.

Head unit 102 may include one or more cameras and one or more image processing units configured to convert the light received from the target area to an image/video. For example, head unit 102 may include a camera configured to receive light from the target area via scope 150 and head unit 102 may include an image processing unit configured to convert the received light to image/video data. The image/video data may be sent to a remote receiver and the remote receive may be coupled to a display configured to convert the image/video data to one or more images/videos for viewing. In some embodiments, device 100 includes a display screen for displaying the image/video of the target area. For example, device 100 may include a display screen disposed on an exterior surface (e.g., housing 105) of device 100 configured to display the image/video of the target area. User interface 116 may include a display screen configured to display the image/video of the target area. In some embodiments, user interface 116 is a touchscreen allowing a user to control head unit 102 and also view images/video of the target area. Alternatively, device 100 may transmit image/video data to an external display. In some embodiments, device 100 wirelessly transmits image/video data to an external display.

In some embodiments, head unit 102 is sized and shaped to fit within the hand of user. For example, head unit 102 may have an ergonomic design that allows head unit 102 to fit easily in the hand of individual users for extended periods of time without causing discomfort. Head unit 102 may be configured to offer multiple gripping and holding options. In some embodiments, head unit 102 allows device 100 to be lighter in weight compared to traditional imaging devices. For example, head unit 102 may be 300 grams to 500 grams, 325 grams to 450 grams, or 350 grams to 400 grams. For example, head unit 102, and thus device 100, being wireless reduces the need for additional wires and cables thereby reducing the overall weight of device 100 as well as eliminating the effects of wires and cables pulling on head unit 102.

With continued reference to FIGS. 1-4, head unit 102 may include housing 105. Housing 105 may comprise the external surface of head unit 102. In some embodiments, housing 105 is comprised of a durable material to allow for more rugged use of device 100. For example, housing 105 may be comprised of a durable polymer, metal, or combination thereof to allow head unit 102 to be dropped or receive one or more impacts without damaging head unit 102. Housing 105 may be made via molding, casting, reductive processes, and/or an additive process (e.g., 3D printing). Housing 105 may include internal cavity 118, which may be configured to receive battery 108. Battery 108 may be a removable battery configured to be inserted into and removed from internal cavity 118.

In some embodiments, internal cavity 118 is configured to receive battery 108 in a specific orientation. Internal cavity 118 may include connecting element 119 configured to couple battery 108 to head unit 102. In some embodiments, fully inserting battery 108 into internal cavity 118 results in coupling battery 108 to connecting element 119. Coupling of battery 108 and connecting element 119 may result in battery 108 being coupled to head unit 102 and battery 108 providing power to head unit 102. For example, inserting battery 108 into internal cavity 118 such that battery 108 couples to connecting element 119 may result in battery 108 providing power to head unit 102.

Referring to FIG. 1, battery 108 may include proximal end 107 and distal end 109. Distal end 109 may be opposite proximal end 107. In some embodiments, when battery 108 is fully inserted into internal cavity 118, distal end 109 may remain exposed. Distal end 109 being exposed may allow a user to easily grip onto battery 108 and remove it from internal cavity 118 to replace, repair, or recharge battery 108. In some embodiments, when battery 108 is fully inserted into internal cavity 118, distal end 109 is flush with housing 105 to allow a user to comfortably grip head unit 102 proximate distal end 109 of battery 108 during use of device 100.

In some embodiments, head unit 102 includes proximal end 101 and distal end 103. Proximal end 101 may be opposite distal end 103 and may include power cable port 120 and mount 115. In some embodiments, each of power cable port 120 and mount 115 are opposite internal cavity 118. For example, power cable port 120 and mount 115 may be disposed on proximal end 101, which may be disposed opposite from internal cavity 118, which is disposed on distal end 103. Proximal end 101 may include a planar front face. In some embodiments, power cable port 120 and mount 115 are disposed on the planar front face such that power cable port 120 is co-planar with mount 115.

In some embodiments, proximal end 101 is substantially planar such that power cable port 120 is co-planar with mount 115. Power cable port 120 may be coupled to power cable 112 thereby coupling power cable 112 to head unit 102. Power cable port 120 may be coupled to proximal end 113 of power cable 112. In some embodiments, power cable 112 is fixedly coupled to power cable port 120. However, power cable 112 may be removably coupled to power cable port 120.

In some embodiments, proximal end 101 includes mount 115. Mount 115 may be configured to couple to scope 150 (e.g., endoscope). In some embodiments, mount 115 is configured to removably coupled head unit 102 to scope 150. Mount 115 may be configured to be compatible with different types of coupling mechanisms. For example, mount 115 may be compatible with C-mount couplers, eye-piece couplers, direct view couplers, or any other type of coupling mechanism. In some embodiments, mount 115 is configured to allow head unit 102 to couple to C-mount scopes, eye-piece scopes, direct view scopes, or any other type of scope. Mount 115 may be configured to couple to and secure scope 150 to head unit 102. Mount 115 may allow head unit 102 to be plug-and-play compatible with various types of scopes, such as scope 150. For example, during use of device 100, a user may couple a first scope to head unit 102 (e.g., via mount 115) and then easily decouple the first scope from head unit 102 and couple a second scope to head unit 102. In some embodiments, scope 150 includes adjustment mechanism 114 and scope 150 is coupled to mount 115 via adjustment mechanism 114. Adjustment mechanism 114 may assist in securing scope 150 to head unit 102 at mount 115. In some embodiments, adjustment mechanism 114 allows scope 150 to be freely rotated relative to head unit 102 without disengaging scope 150 from mount 115. In some embodiments, scope 150 is configured to rotate 360 degrees relative to head unit 102 and/or mount 115 to allow a user to achieve the desired orientation and view.

In some embodiments, scope 150 includes adjustment mechanism 114 such that scope 150 is coupled to mount 115 via adjustment mechanism 114. Alternatively, head unit 102 may include adjustment mechanism 114 coupled to mount 115 and scope 150 may couple to adjustment mechanism 114 to secure scope 150 to mount 115 and head unit 102.

In some embodiments, adjustment mechanism 114 is configured to allow a user to adjust and/or control various functions of device 100. For example, adjustment mechanism 114 may allow a user to adjust the focus and/or zoom (e.g., zooming in and out) of the images/videos received by head unit 102 from scope 150. Adjustment mechanism 114 may also allow a user to adjust the amount of light emitted by scope 150 to the target area. In some embodiments, during use of device 100, a user may hold head unit 102 with a single hand and use the same hand to adjust adjustment mechanism 114. Alternatively, a user may use a different hand to adjust adjustment mechanism 114.

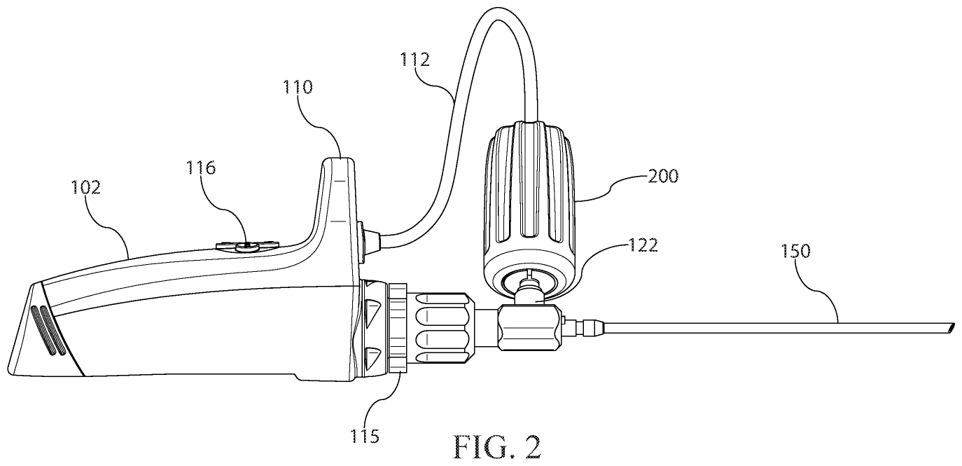

Referring to FIGS. 1-2, proximal end 101 may include protrusion 110. Protrusion 110 may be configured to prevent a user's hand from slipping towards scope 150. In some embodiments, protrusion 110 provides an ergonomic resting location for a user's hand or a portion of the user's hand. Protrusion 110 may include one or more antennas. The one or more antennas may be configured to transmit data (e.g., image/video data, command and control signals, etc.) to a remote receiver and receive information from a remote device. In some embodiments, the one or more antennas are disposed anywhere within head unit 102, such as proximate proximal end 101, proximate distal end 103, and/or between proximal end 101 and distal end 103.

In some embodiments, the one or more antennas are configured to wirelessly communicate with a remote receiver (e.g., receiver 400). For example, head unit 102 may include one or more antennas and head unit 102 may be configured to wirelessly communicate with a remote receiver via a wireless modality. The wireless modality may be ultrawide band (UWB), Bluetooth, Nearfield communication (NFC), Wi-Fi, Cellular, or any other type of wireless modality. In some embodiments, device 100 utilizes UWB to communicate with a remote receiver (e.g., receiver 400). Device 100 may further include built in encryption to securely transmit data to the remote receiver. For example, device 100 may associate the image/video data generated by head unit 102 with an authentication factor, such as an authentication token. Device 100 may transmit the image/video data with the authentication factor to a remote receiver (e.g., receiver 400). The remote receiver may include an encryption engine configured to authenticate the authentication factor received with the image/video data to ensure that the image/video data was transmitted securely from head unit 102. Further, associated the image/video data generated by head unit 102 with an authentication factor prevents unauthorized users and/or devices from accessing the image/video data.

In some embodiments, during use of device 100, other devices, such as other wireless devices, are simultaneously used adjacent to device 100, which results in interference. Device 100 utilizing UWB for wireless communication prevents or minimizes interference from other devices while maintaining the ability to send large packets of data (e.g., image/video data). For example, utilizing UWB communication allows device 100 to send large sized image/video data containing high resolution images/videos to the remote receiver (e.g., receiver 400) for viewing via a display coupled to the receiver, as well as command and control signals. In some embodiments, wireless communication between device 100 and a remote receiver is configured to act as a cable for coding purposes. This allows for the simplification of communication protocols and debugging of issues during use of device 100. In some embodiments, device 100 is configured to provide low latency communication such that lag is minimized between head unit 102 receiving data from the target area being viewed by scope 150 and what is being received by the receiver and displayed to the user via a display screen. Device 100 providing low latency communication increases the efficiency of procedures during use of device 100 in addition to reducing injuries, mistakes, or accidents during procedures using device 100.

In some embodiments, distal end 103 is configured to receive battery 108 via internal cavity 118. In some embodiment, head unit 102 is coupled to battery 108 to allow device 100 to be wireless without the need for power lines. Battery 108 may be rechargeable. In some embodiments, battery 108 is configured to be removably coupled to head unit 102 such that battery 108 is able to be quickly swapped out and replaced with a fully charged battery 108. In some embodiments, when battery 108 is received in internal cavity 118 of head unit 102, all or a portion of distal end 109 (e.g., a distal portion) may be exposed (e.g., a portion of distal end 109 not disposed within internal cavity 118) is flush with housing 105. Head unit 102 may include locking device 117 to secure battery 108 in place when battery 108 is coupled to head unit 102. Locking device 117 may be configured to allow battery 108 to be removably coupled to head unit 102. For example, locking device 117 may be configured to engage with a portion of battery 108 to secure battery 108 within internal cavity 118. A user interact with locking device 117, such as disengaging locking device 117, to allow for the removal of battery 108 from internal cavity 118. In some embodiments, locking device 117 allows battery 108 to be quickly changed and reset in approximately 30 seconds. Battery 108 and locking device 117 reduces the down time during replacing of battery 108 during use of device 100.

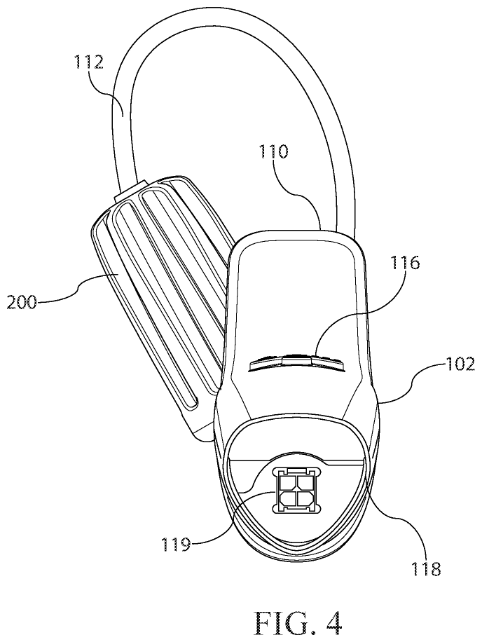

Referring to FIG. 4, replacing battery 108 within head unit 102 results in resetting device 100 to a base state. For example, when battery 108 is fully disposed within internal cavity 118, battery 108 may engage with connecting element 119. When battery 108 is at least partially removed from internal cavity 118, battery 108 may no longer engage with connecting element 119 thereby causing device 100 to reset and/or enter a base state. Resetting device 100 to a base state may assist in fixing any errors or issues that arise during use of device 100. In resetting device 100 to a base state, all or most settings of device 100 may be reset to address any errors or issues that previously arose. In addition, resetting device 100 ensures that no protected data, such as patient or HIPAA data, is stored locally, and allows for a quick boot-up cycle.

In some embodiments, device 100 includes an internal battery disposed within head unit 102. The internal battery may provide power to device 100 when battery 108 disengages from connecting element 119 and/or is removed from internal cavity 118. The internal battery providing power to device 100 may prevent device 100 from resetting or entering a base state when battery 108 is replaced or removed. This allows for continuous use of device 100 even when battery 108 is being replaced or is removed. In some embodiments, device 100 is configured to allow a user to disengage the internal battery to prevent the internal battery from providing power to device 100 during replacing and/or removing of battery 108. Disengaging the internal battery allows a user to reset device 100 to a base state when battery 108 is disengaged from connecting element 119 and/or when battery 108 is removed from internal cavity 118.

In some embodiments, battery 108 is configured to provide at least 1 hour of continuous run time for device 100. For example, single use of battery 108 may result in at least 1 hour, 2 hours, 3 hours, 4 hours, 5 hours, or greater than 6 hours of run time of device 100. In some embodiments, battery 108 is configured to provide a runtime of 30 minutes to 600 minutes, 180 minutes to 540 minutes, or 240 minutes to 480 minutes. Battery 108 may be configured to allow device 100 to be operated without a battery change/replacement for longer than most surgeries or procedures require. This is due to the power and efficiency of battery 108 and very efficient electronics of device 100, which are designed to ensure longevity of battery 108 and use of device 100. Further, device 100 may be configured to operate with lower power consumption than traditional imaging devices. For example, device 100 may require less energy to operate compared to traditional imaging devices. In some embodiments, device 100 uses 1% to 5% of the total power required by traditional imaging devices. Wireless imaging device system, including device 100, receiver 400, and battery charging device 300, uses 1% to 5% of the total power required by traditional imaging systems. This results in less electricity usage thereby resulting in lower expenses for users of device 100 (e.g., medical provider or hospital). Further, device 100 being wireless and portable results in device 100 having a smaller footprint compared to traditional imaging devices. Device 100 having a smaller footprint allows for easier cleaning and sterilization of device 100 compared to traditional imaging devices.

In some embodiments, the reduced power usage of our wireless imaging device system (device 100, receiver 400, and battery charging device 300) also reduces the amount of waste heat generated compared to traditional imaging systems. The waste heat must be removed from the OR, leading to increased power savings. Wireless imaging device system (device 100, receiver 400, and battery charging device 300) reducing the amount of waste heat results in a more efficient imaging system due to decreased electricity usage (heating, ventilation, and air condition).

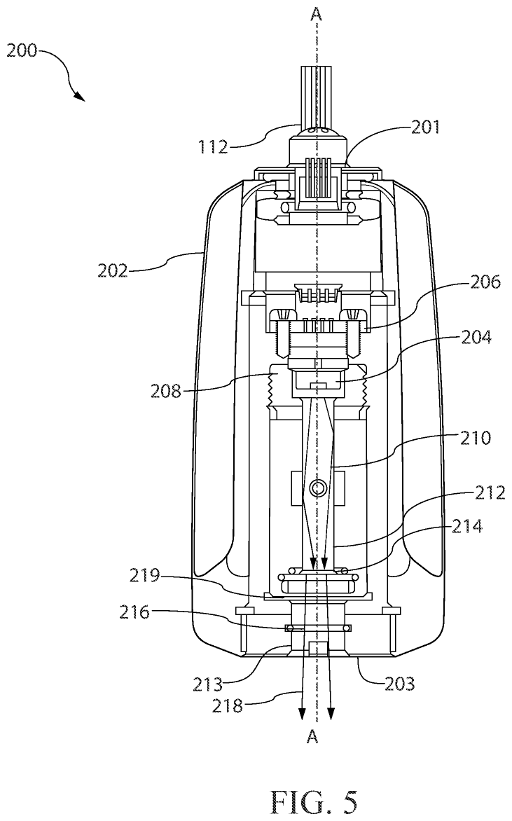

Referring to FIGS. 1 and 5, head unit 102 may be coupled to light engine 200 via power cable 112. Light engine 200 may include first end 201 and second end 203. First end 201 may be disposed opposite second end 203. In some embodiments, light engine 200 is coupled to power cable 112 at first end 201 and is coupled to scope 150 at second end 203. First end 201 and second end 203 may be disposed along central axis A-A. In some embodiments, central axis A-A bifurcates light engine 200. For example, light engine 200 may be symmetrical about central axis A-A.

Power cable 112 may include proximal end 113 and distal end 111. In some embodiments, proximal end 113 is coupled to head unit 102 at power cable port 120. Distal end 111 may be coupled to light engine 200. Power cable 112 may be configured to provide power to light engine 200. In some embodiments, power cable 112 is flexible to allow light engine 200 to rotate relative to head unit 102. For example, power cable 112 being flexible may allow for 360 degrees rotation of light engine 200 relative to head unit 102. In some embodiments, power cable 112 is configured to be flexible to prevent impeding the rotation of light engine 200 relative to head unit 102. Light engine 200 may be configured to generate light for illuminating a target area. Light engine 200 may output light that travels through scope 150 to the target area.

Light engine 200 may be configured to generate and output light. Light engine 200 may include outer shell 202. Outer shell 202 may be comprised of aluminum or another metal or polymer with high heat transfer. In some embodiments, outer shell 202 serves as a heat sink to dissipate heat generated by light engine 200. For example, outer shell 202 may dissipate heat generated by light engine 200 to cause light engine 200 to stay below a temperature that may cause burn injuries, such as below approximately 43.5° C. However, outer shell 202 may be configured to dissipate heat generated by light engine 200 to cause light engine 200 to stay below approximately 45° C., approximately 40° C., approximately 35° C., approximately 30° C., or approximately 25° C. In some embodiments, light engine 200 is configured to stay below approximately 45° C. (e.g., below 43.5° C.) during extended use of device 100 (e.g., use exceeding 4 hours). Light engine 200, and by extension device 100, staying below approximately 40° C. prevents burn injuries to individuals around device 100 in addition to preventing device 100 from becoming a fire hazard.

In some embodiments, light engine 200 includes illumination source 204. Illumination source 204 may be a laser, a light emitting diode (LED), incandescent light sources, or any other type of sources capable of providing illumination. Illumination source 204 may be coupled to power cable 112 via circuit board 206. In some embodiments, circuit board 206 is a printed circuit board disposed within outer shell 202 of light engine 200. Circuit board 206 may be configured to communicate with head unit 102 via power cable 112. In some embodiments, circuit board 206 controls the amount of light generated by illumination source 204 and/or the amount of light outputted by light engine 200.

Light engine 200 may include holder assembly 208, which may be configured to secure illumination source 204 in place. In some embodiments, holder assembly 208 acts a heat sink to dissipate heat generated by illumination source 204. Holder assembly 208 may be configured to secure illumination source 204 in place during movement of light engine 200 and/or device 100.

Illumination source 204 may be configured to generate and output light 210. Illumination source 204 may output light 210 having a peak wavelength of 440 nm to 470 nm. In some embodiments, light 210 is unconditioned light outputted by illumination source 204. Light engine 200 may include optical element 212 configured to condition, collimate, filter, direct, and/or focus light 210 outputted by illumination source 204. In some embodiments, optical element 212 includes one or more homogenizing rods configured to collimate and direct light 210. Optical element 212 may be comprised of glass, plastic, or any other type of material to allow optical element 212 to condition, collimate, filter, direct, and/or focus light 210. Optical element 212 may be configured to direct and/or collimate light 210 onto converter 214. Converter 214 may be disposed within outer shell 202 of light engine 200 and may be configured to convert light 210 to light 218. In some embodiments, light 218 has a different wavelength than light 210.

In some embodiments, light converter or converter 214 is configured to convert incoming light 210 to light 218, which is outputted by light engine 200. Light 218 may be white light, between 480 nm-770 nm. In some embodiments, converter 214 is a volumetric spectrum converter comprising phosphor particles. Converter 214 may include phosphor particles volumetrically disposed within a substrate. In some embodiments, the substrate may be a homogenous composite substrate of non-converting material (e.g., plastic, acrylic, glass, ceramic). The phosphor particles being volumetrically disposed within converter 214 allows converter 214 to more efficiently convert light 210 to light 218. In some embodiment, converter 214 is comprised of a plurality of layers and each layer may have a plurality of phosphor particles volumetrically disposed within. The plurality of layers may form a volumetric spectrum converter. In some embodiments, the plurality of layers are arranged such that each layer is substantially perpendicular to light 210. In some embodiments, light 210 strikes converter 214 at an angle of 45 degrees to 135 degrees, 65 degrees to 125 degrees, or 75 degrees to 115 degrees.

Converter 214 may be chosen to convert light from illumination source 204 to light of another wavelength, for example, narrow or broad spectrum, or non-coherent light. Converter 214 may be comprised of a converting material that may include, for example, phosphorescent material, florescent material, other radiation converting material, or combinations of these materials. The converting material is volumetrically disposed in a substrate that may include, for example, PMMA, polystyrene, polycarbonate, polyester, copolymers, or blends of a combination of the aforementioned materials to create an effectively homogenous composite. This process may include, for example, extrusion, coating, lamination, blending, mixing, or suspending. In some embodiments, converter 214 has a thickness of 0.1 mm to 5 mm and a radius of 0.1 mm to 5 mm.

In some embodiments, converter 214 is a volumetric phosphor disk configured to convert light 210 to light 218. Alternatively, converter 214 may be a volumetric phosphor sphere, cube, rectangular prism, pyramid, sphere, or any other shape desired. Converter 214 may be secured within light engine 200. For example, converter 214 may be disposed between optical element 212 and window 219 of light engine 200. In some embodiments, converter 214 is a disk secured between optical element 212 and window 219 via friction, adhesives, epoxy, fasteners, or any other methods of securing converter 214 between optical element 212 and window 219. Light 218 may exit converter 214 such that light 218 passes through window 219 and exits light engine 200. Window 219 may be transparent to allow light 218 to pass through.

In some embodiments, outer shell 202 includes opening 213 that extends at least partially through outer shell 202. Opening 213 may be in optical communication with window 219 such that light 218 from converter 214 passes through window 219 and out of light engine 200 via opening 213. In some embodiments, opening 213 extends through outer shell 202 along central axis A-A. In some embodiments, illumination source 204, optical element 212, and window 219 are aligned. For example, illumination source 204, optical element 212, window 219, converter 214 and opening 213 may be disposed along central axis A-A of light engine 200. In some embodiments, illumination source 204, optical element 212, window 219, converter 214 and opening 213 are arranged in series along central axis A-A.

Referring to FIGS. 2 and 5, light engine 200 may include rings 216. Rings 216 may be configured to assist in coupling light engine 200 to scope 150. For example, scope 150 may include coupling element 122, which may be received by light engine 200 and secured to light engine 200 via rings 216. In some embodiments, scope 150 includes coupling element 122 that is received by rings 216 of light engine 200 such that coupling element 122 and scope 150 are secured to light engine 200. Light engine 200 may be secured to scope 150 via coupling element 122 and scope 150 such that light engine 200 and scope 150 are rotatable relative to head unit 102. For example, light engine 200 and scope 150 may rotate 360 degrees relative to head unit 102.

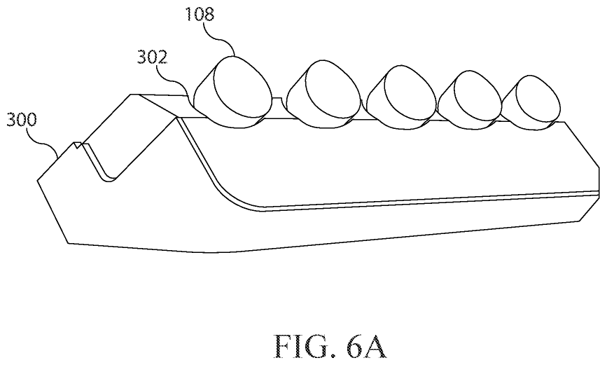

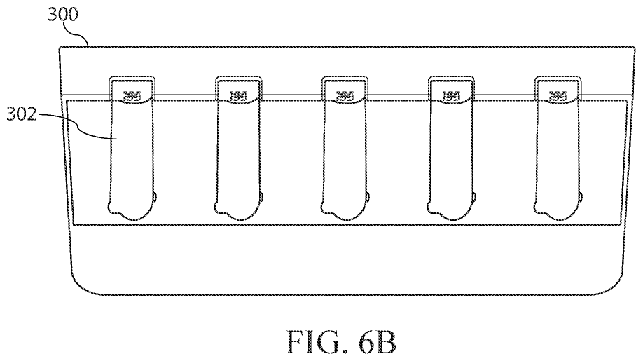

Referring to FIGS. 6A and 6B, battery 108 may be rechargeable and may be recharged using battery charging device 300. Battery charging device 300 may be configured to charge one or more batteries 108. In some embodiments, battery charging device 300 includes one or more grooves 302 configured to receive battery 108. Groove 302 may be sized and shaped to receive and secure battery 108. In some embodiments, groove 302 receives battery 108 to allow battery 108 to rest within battery charging device 300 while battery 108 is being charged. Groove 302 may allow for easy alignment of battery 108 within battery charging device 300 to allow a user to easily charge battery 108. For example, groove 302 may allow a user to easily align battery 108 within battery charging device 300 to quickly initiate charging of battery 108.

Groove 302 may also prevent damage to battery 108 when placing battery 108 within battery charging device 300. Groove 302 may also prevent non-compatible batteries from being used with battery charging device 300. In some embodiments, battery charging device 300 is configured to charge one or more batteries 108 via inductive charging. For example, placing battery 108 within groove 302 of battery charging device 300 may result in battery 108 being charged by battery charging device 300. Alternatively, battery charging device 300 is configured to charge one or more batteries 108 via conductive charging. For example, placing battery 108 within groove 302 of battery charging device 300 causes a battery contact of battery 108 to contact or abut a charging contact of battery charging device 300 resulting in battery charging device 300 charging battery 108.

Battery 108 may also be configured to be easily sterilizable. For example, battery 108 may be configured to be easily sterilized via one or more vapors, gases, or solutions. In some embodiments, battery 108 is configured to be sterilizable with a vapor such as vaporized hydrogen peroxide (VHP). In some embodiments, battery 108 is easily VHP sterilizable after being charged. This simplifies and shortens the reprocessing procedure saving time and money.

In some embodiments, head unit 102 and/or battery 108 are configured to be single use. For example, head unit 102 and/or battery 108 may be disposable such that after use of head unit 102, the user discards one or more of head unit 102 and battery 108. In some embodiments, head unit 102 and/or battery 108 are configured to be disposed within a sterilization pouch. For example, prior to use, a user may have to remove head unit 102 and/or battery 108 from the sterilization pouch. Head unit 102 and/or battery 108 may be pre-sterilized prior to being placed into the sterilization pouch. The sterilization pouch may be configured to keep head unit 102 and/or battery 108 sterilized until a user opens and removes head unit 102 and/or battery 108 from the sterilization pouch for use.

Referring to FIGS. 1-4, head unit 102 may include user interface 116. User interface 116 may be disposed between proximal end 101 and distal end 103 of head unit 102. For example, user interface 116 may be disposed on housing 105, such as an external surface of head unit 102. In some embodiments, user interface 116 includes one or more buttons. However, user interface 116 may be a touchscreen, scrolling wheel, slider, or any other type of user interaction element. In some embodiments, user interface 116 is located on housing 105 such that a user can easily access user interface 116 via one or more fingers during use of device 100. For example, a user may grip or hold head unit 102 using a single hand and may interact with user interface 116 using fingers of the same hand.

In some embodiments, user interface 116 may allow a user to control various functions of device 100. A user may interact with user interface 116 via one or more buttons, a touch screen, a scrolling wheel, a slider, or other type of interaction element to allow a user to control various features of device 100. For example, a user may interact with user interface 116 to adjust the zoom, focus, brightness, saturation, resolution, contrast, white balance, and/or exposure level of the image/video generated by head unit 102 or transmitted by head unit 102 to an externa display device, such as via a receiver (e.g., receiver 400). In some embodiments, user interface 116 is configured to allow a user to control and/or adjust the amount of light emitted from light engine 200 and received by head unit 102. For example, a user may use user interface 116 to control the amount of light that is emitted from light engine 200 and that illuminates the target area. A user may also use user interface 116 to control the amount of light captured by a camera disposed within head unit 102. In some embodiments, user interface 116 allows a user to capture images and/or videos of the target area and select a storage location (e.g., internal storage, external storage device).

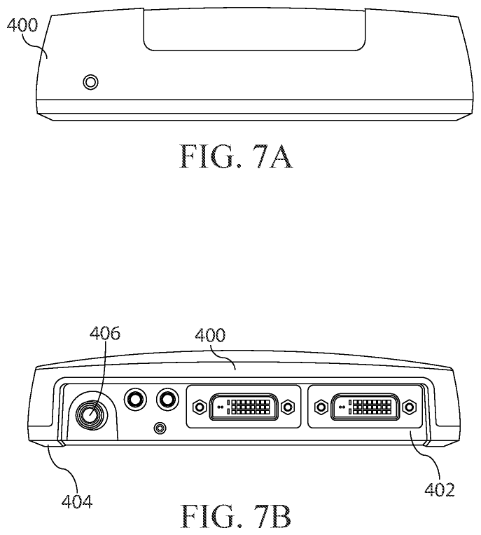

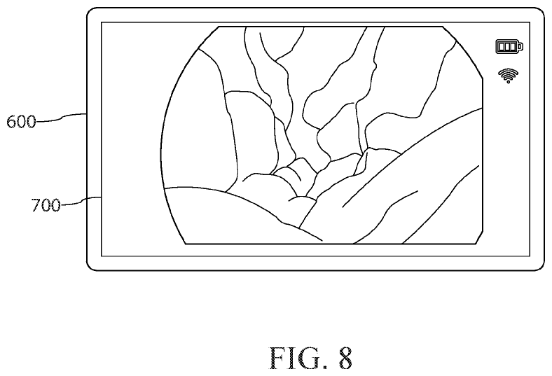

Referring to FIGS. 7A-9A, device 100 may be configured to wirelessly communicate with receiver 400. Receiver 400 may be configured to receive data (e.g., image/video data, command and control signals, etc.) from device 100. Receiver 400 may have a considerably smaller footprint compared to receivers of traditional imaging devices. In some embodiments, receiver 400 can be scaled in size depending on the desired use and location of use. For example, receiver 400 may be scaled down to be smaller to allow for easier portability. In some embodiments, receiver 400 is configured to wirelessly receive encrypted data from head unit 102 and wirelessly transmit that encrypted data to a display device for viewing images/videos associated with the encrypted data. Alternatively, receiver 400 may be coupled to an external display device and may transmit the encrypted data to the external display device for viewing images/videos associated with the encrypted data.

Receiver 400 may include back panel 402. Back panel 402 may include one or more ports 406 for coupling to one or more devices. For example, back panel 402 may include one or more ports for coupling to a display device (e.g., display device 600). In practice, head unit 102 of device 100 is configured to transmit image/video data or command and control signals to receiver 400. Head unit 102 may transmit data (e.g., encrypted data) to receiver 400 via UWB. In some embodiments, head unit 102 transmits images or videos of a target area to receiver 400. In additional embodiments, head unit 102 transmits other types of data to receiver 400, such as command and control signals. Receiver 400 may be coupled to a display device to allow one or more users to view the images or videos of the target area captured by head unit 102.

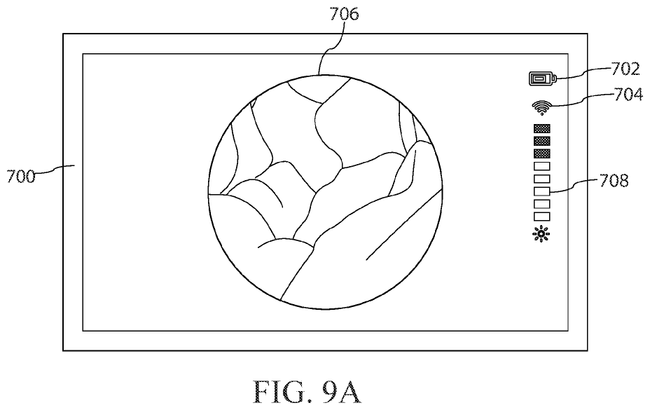

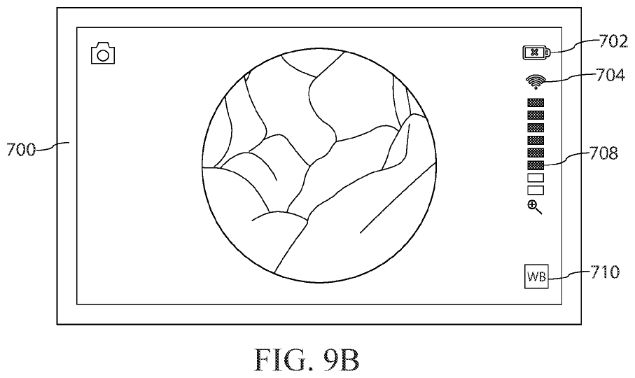

In some embodiments, receiver 400 is configured to receive images/video data in real time, with low latency, and is configured to cause display device 600 to display the images/videos to allow a user to view the illuminated target area in real time, with low latency. Display device 600 may be configured to generate display 700. Display 700 may be in color (FIG. 8), black and white (FIGS. 9A and 9B), or grey scale.

In some embodiments, receiver 400 causes display device 600 to display and generate display 700. For example, receiver 400 may receive image/video data from head unit 102 and may transmit the image/video data to display device 600 for displaying of images/videos associated with the image/video data. Display 700 may be configured to display image 706, which may be an image received by scope 150, and processed and transmitted to receiver 400 by head unit 102. For example, image 706 may be an image of the target area where scope 150 is viewing. In some embodiments, display 700 is configured to generate and display images or videos. Display 700 may include power indicator 702 to indicate the power level of battery 108. Display 700 may include signal indicator 704 to indicate the strength of the wireless connection between head unit 102 and receiver 104. For example, signal indicator 704 may indicate the strength of the UWB connection between head unit 102 and receiver 104.

In some embodiments, display 700 includes brightness indicator 708. Brightness indicator 708 may indicate the brightness level of image 706. In some embodiments, brightness indicator 708 indicates the lux of light 218 from light engine 200. For example, a user may use user interface 116 to adjust the lux of light engine 200, thereby controlling the brightness of light 218, resulting in image 706 being brighter or dimmer. The increase in brightness may be illustrated via brightness indicator 708.

Display 700 may include function indicator 710. In some embodiments, function indicator 710 is a white balance function indicator to indicate the activation of a white balance function. For example, display 700 may have a low brightness level resulting in image 706 being faint or having low brightness making it difficult to ascertain the features of the target area viewed by scope 150. In some embodiments, user interface 116 is used to increase the brightness of display 700. For example, a user may use user interface 116 to increase the brightness of display 700 resulting in brightness indicator 708 showing an increase in the brightness level of image 706. In some embodiments, brightness indicator 708 may indicate the change to the sensitivity to the image or video sensor disposed within head unit 102. This may indicate how bright an image is outputted by head unit 102. For example, a user may use user interface 116 to increase the brightness of image 706 resulting in a change to the sensitivity of the image or video sensor of head unit 102. An increase in the brightness of image 706 results in a change to brightness indicator 708.

Additionally, display 700 may have colors that are incorrectly displayed making it difficult to ascertain the features of the target area viewed by scope 150. Function indicator 710 may indicate that a white balance function is being applied to image 706 resulting in white objects in image 706 appearing brighter or more color accurate to assist the user in seeing features of image 706.

In some embodiments, display 700 is configured to digitally zoom into image 706. For example, a user may use user interface 116 to zoom in and zoom out of image 706 via display 700. In some embodiments, display 700 is configured to automatically zoom in or out based on the contents of image 706. A user may use user interface 116 or display 700 to digitally zoom in and zoom out of image 706.

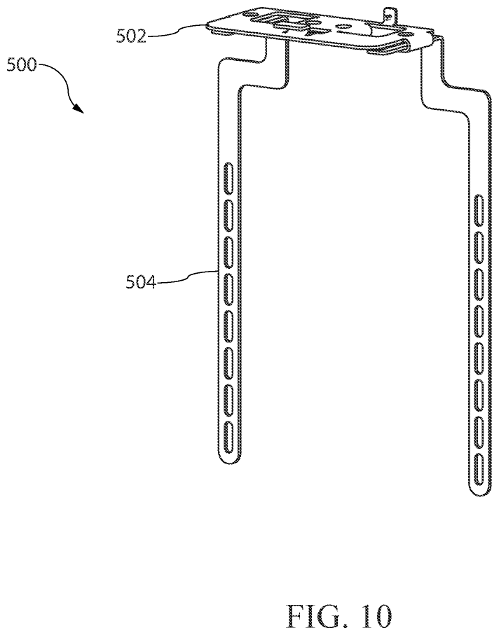

Referring to FIGS. 7A-7B and 10, receiver 400 may be configured to couple with mounting bracket 500. Mounting bracket 500 may be configured to allow receiver 400 to couple to a display (e.g., display device 600) such that receiver 400 is secured to a display device. For example, mounting bracket 500 may be coupled to bottom 404 of receiver 400 and mounting bracket 500 may further be coupled to display device 600 to couple receiver 400 to display device 600. Mounting bracket 500 may include one or more arms 504 and coupling mechanism 502. Coupling mechanism 502 may be configured to couple to receiver 400 (e.g., bottom 404 of receiver 400). Arm 504 may be configured to assist mounting bracket 500 in coupling to display device 600. In some embodiments, coupling of arm 504 to display and coupling mechanism 502 to receiver 400 results in receiver 400 being coupled to and secured to display device 600. In some embodiments, mounting bracket 400 has two arms 504. However, mounting bracket 500 may have one arm, three arms, four arms, or greater than four arms. In some embodiments, coupling arms 504 are intended to interface with a VESA mount, or other mounting standard, on the back of a display device 600.

In some embodiments, mounting bracket 500 is configured to couple to receiver 400 and to the back of display device 600 such that receiver 400 is disposed on top of display device 600. Receiver 400 being coupled to display device 600 such that it sits on top of display device 600 results in receiver 400 and the display having an overall smaller footprint and results in a less bulky configuration. Further, receiver 400 being disposed on top of display device 600 reduces the amount of cluttering and loose cords. In some embodiments, display device 600 is a portable display to allow for the use of receiver 400, and device 100, at any location.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. The words “front”, “back”, “inward” and “outward” designate directions in the drawings to which reference is made. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

What is claimed is:

1. A wireless imaging device comprising:
a head unit configured to couple to a scope, the head unit having a housing with an internal cavity configured to receive a battery;
a flexible cable having a proximal end and a distal end, the proximal end of the flexible cable being coupled to the head unit; and
a light engine having a first end coupled to the distal end of the flexible cable and a second end opposite the first end, the light engine configured to be removably coupled to a scope at the second end such that when the light engine is coupled to the scope, the light engine and the scope are freely rotatable relative to the head unit.

2. The wireless imaging device of claim 1, wherein the light engine includes a light converter comprising a plurality of phosphor particles volumetrically disposed within a substrate.

3. The wireless imaging device of claim 2, wherein the light engine includes an optical element and a window such that the light converter is disposed between the optical element and the window of the light engine.

4. The wireless imaging device of claim 2, wherein the light converter is a disk.

5. The wireless imaging device of claim 1, wherein the head unit includes a first end and a second end opposite the first end, the first end having a planar front face.

6. The wireless imaging device of claim 5, wherein the planar front face includes a power cable port and a mount, the power cable port being co-planar with the mount and coupled to the flexible cable.

7. The wireless imaging device of claim 5, wherein the planar front face is disposed opposite the internal cavity.

8. The wireless imaging device of claim 1 further comprising:
a removable battery sized and shaped to be inserted into the internal cavity.

9. The wireless imaging device of claim 8, wherein the removable battery includes an exposed distal portion that is exposed and flush with the housing when the removable battery is disposed within the internal cavity.

10. The wireless imaging device of claim 8, wherein the head unit includes a coupling element configured to engage the removable battery when the removable battery is inserted into the internal cavity.

11. The wireless imaging device of claim 1, wherein the head unit includes a mount configured to couple to one or more of C-mount scopes, eye-piece scopes, and direct view scopes.

12. The wireless imaging device of claim 1, wherein the head unit includes a display screen configured to display an image of a target area viewed by the scope.

13. The wireless imaging device of claim 1, wherein the light engine includes a volumetric converter, an optical element, a window, and an opening arranged in series along a central axis.

14. The wireless imaging device of claim 1, wherein the light engine includes an outer shell, the outer shell configured to maintain the light engine at a temperature under approximately 45° C.

15. The wireless imaging device of claim 1 further comprising:
at least one antenna disposed within the head unit, the at least one antenna configured to wirelessly communicate with a remote receiver.

16. The wireless imaging device of claim 1, wherein the light engine includes an illumination source generating a first light having a first wavelength, the first light being converted by a volumetric converter to a second light having a second wavelength different than the first wavelength.

17. The wireless imaging device of claim 1, wherein the light engine includes an outer shell and an opening extending at least partially through the outer shell along a central axis.

18. A wireless imaging device comprising:
a head unit configured to couple to a scope, the head unit having a housing with an internal cavity configured to receive a battery, a first end having a planar face including a power cable port and a mount configured to receive a scope, and a second end being opposite the first end and having the internal cavity;
a flexible cable having a proximal end and distal end, the proximal end of the flexible cable being coupled to the head unit at the power cable port; and
a light engine coupled to the distal end of the flexible cable, the light engine being configured to be removably coupled to the scope such that when the light engine is coupled to the scope, the light engine and the scope are freely rotatable relative to the head unit,
wherein the light engine includes a volumetric converter, an optical element, a window, and an opening arranged in series along a central axis, and
wherein the light engine includes an illumination source generating a first light having a first wavelength, the first light being converted by the volumetric converter to a second light having a second wavelength different than the first wavelength.

19. An imaging system comprising:
a wireless imaging device having a head unit configured to removable couple to a scope, the head unit including a proximal end and distal end, the proximal end of the head unit including a power cable port coupled to a flexible cable and the distal end of the head unit including an internal cavity configured to receive a battery, the wireless imaging device including a light engine having a first end coupled to the head unit via the flexible cable and a second end opposite the first end configured to couple to the removable scope, wherein the wireless imaging device includes one or more antennas disposed within the head unit; and
a receiver disposed remote to the wireless imaging device, the receiver being in wireless communication with the one or more antennas and configured to wirelessly receive imaging data from the one or more antennas.

20. The imaging system of claim 19 further comprising:
a display device in communication with the receiver, the display device having a display screen configured to display the imaging data.
